(19) United States
(12) Patent Application Publication
(10) Pub. No.: US 2026/0263739 A1
(43) Pub. Date: Sep. 10, 2026

(54) URINARY CATHETER WITH CLOSABLE DRAINAGE OPENINGS

(71) Applicant: Hollister Incorporated, Libertyville, IL (US)

(72) Inventor: David J. Farrell, Ballina (IE)

(21) Appl. No.: 19/164,496

(22) PCT Filed: Mar. 12, 2024
(86) PCT No.: PCT/US2024/019590

§ 371 Date: Sep. 11, 2025

Related U.S. Application Data
(60) Provisional application No. 63/490,628, filed on Mar. 16, 2023.

(51) Int. Cl. A61M 25/00

(52) U.S. Cl. CPC A61M 25/0017

(57) ABSTRACT

Urinary catheters (10) that include drainage openings (22) and closure elements (24) that close the drainage openings during advancement and withdrawal of the urinary catheter from the urethra and open the drainage openings in the bladder.

The present application claims the benefit of and priority to U.S. Provisional Application No. 63/490,628, filed Mar. 16, 2023, which is hereby incorporated by reference.

### FIELD OF DISCLOSURE

The present disclosure is generally related to intermittent urinary catheters that have drainage openings or eyelets that move between an open configuration and closed configuration, and more particularly related to drainage openings that have a closed configuration during advancement through the urethra and an open configuration in the bladder.

### BACKGROUND

Intermittent urinary catheters include a catheter shaft or tube having a proximal insertion end portion that is inserted through the urethra and into the bladder. Once in the bladder, urine enters the catheter through one or more drainage openings or eyelets. Urine enters into these drainage opening(s), drains through an inner drainage lumen of the catheter tube and out of a distal drainage opening in the distal end portion of the catheter tube. After urine has been drained out of the bladder, the catheter is withdrawn from the urethra.

It is desirable to reduce the risk of user discomfort during advancement and withdrawal of the catheter tube through the urethra. Therefore, there remains a need for intermittent urinary catheters having features that reduce the risk of discomfort during use.

### SUMMARY

In one aspect, urinary catheter product includes a catheter tube configured to be inserted into a urethra. The catheter tube having a catheter tube proximal insertion end, a catheter tube distal end and a catheter tube internal drainage lumen. The product includes one or more drainage openings associated with the catheter tube proximal insertion end. The one or more drainage openings are in fluid communication with the catheter tube internal drainage lumen and are configured to drain urine from a bladder. The product includes one or more closure elements associated with the one or more drainage openings. The one or more closure elements have an open configuration wherein the one or more drainage openings are exposed and a closed configuration wherein the one or more closure elements close the one or more drainage openings.

### BRIEF DESCRIPTION OF DRAWINGS

FIG. 1 is a top view of an intermittent urinary catheter in accordance with the present disclosure.

FIG. 2 is an enlarged side view of the proximal insertion end of the catheter tube shown in FIG. 1.

FIG. 3 is an enlarged side view of the catheter tube proximal insertion end of FIG. 2 shown located in a portion of the urethra.

FIG. 4 is a cross-sectional view of the catheter tube proximal insertion end shown in FIG. 3 taken along line 4-4.

FIG. 5 is an enlarged side view of the catheter tube proximal insertion end of FIG. 2 shown after the catheter tube tip has entered the bladder and with a segment of the catheter tube proximal insertion end portion located in the urethra adjacent to the bladder.

FIG. 6 is an enlarged side view of the catheter tube proximal insertion end of FIG. 2 shown with the catheter tube proximal insertion end located in the bladder.

FIG. 7 is an enlarged side view of the catheter tube proximal insertion end of FIG. 2 shown after a segment of the catheter tube proximal insertion end has been withdrawn into the urethra.

FIG. 8 is an enlarged side view of the catheter tube proximal insertion end of FIG. 2 shown withdrawn into a portion of the urethra adjacent to the bladder.

FIG. 9 is an enlarged side view of an alternative proximal insertion end of the catheter tube shown in FIG. 1.

FIG. 10 is an enlarged side view of the catheter tube proximal insertion end of FIG. 9 shown located in a portion of the urethra.

FIG. 11 is an enlarged side view of the catheter tube proximal insertion end of FIG. 9 shown after the catheter tube tip has entered the bladder and with a segment of the catheter tube proximal insertion end portion located in the urethra adjacent to the bladder.

FIG. 12 is an enlarged side view of the catheter tube proximal insertion end of FIG. 9 shown with the catheter tube proximal insertion end located in the bladder.

FIG. 13 is an enlarged side view of the catheter tube proximal insertion end of FIG. 9 shown after a segment of the catheter tube proximal insertion end has been withdrawn into the urethra.

FIG. 14 is an enlarged side view of the catheter tube proximal insertion end of FIG. 9 shown withdrawn into a portion of the urethra adjacent to the bladder.

FIG. 15 is an enlarged side view of an alternative proximal insertion end of a catheter tube in accordance with the present disclosure.

FIG. 16 is an enlarged side view of the catheter tube proximal insertion end of FIG. 15 shown being inserted into the urethral opening.

FIG. 17 is an enlarged side view of the catheter tube proximal insertion end of FIG. 15 shown with the catheter proximal end portion located within the urethra.

FIG. 18 is an enlarged side view of the catheter tube proximal insertion end of FIG. 15 shown after the catheter tube tip has entered the bladder and with a segment of the catheter tube proximal insertion end portion located in the urethra adjacent to the bladder.

FIG. 19 is an enlarged side view of the catheter tube proximal insertion end of FIG. 15 shown with the catheter tube proximal insertion end located in the bladder.

FIG. 20 is an enlarged side view of an alternative proximal insertion end of a catheter tube in accordance with the present disclosure.

FIG. 21 is an enlarged side view of the catheter tube proximal insertion end of FIG. 20 shown being inserted into the urethral opening.

FIG. 22 is an enlarged side view of the catheter tube proximal insertion end of FIG. 20 shown with the catheter proximal end portion located within the urethra.

FIG. 23 is an enlarged side view of the catheter tube proximal insertion end of FIG. 20, shown with the catheter tube proximal insertion end located in the bladder.

### DETAILED DESCRIPTION

While the subject matter of the present disclosure is susceptible to embodiments in various forms, there will hereinafter be described presently preferred embodiments with the understanding that the present disclosure is to be considered an exemplification and is not intended to limit the disclosure to the specific embodiments illustrated. The words “a” or “an” are to be taken to include both the singular and the plural. Conversely, any reference to plural items shall, where appropriate, include the singular.

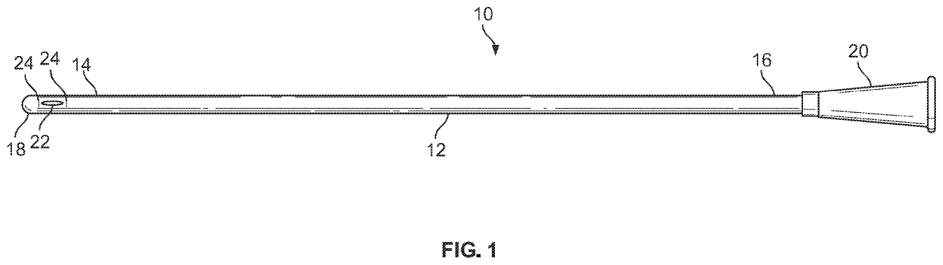

FIG. 1 illustrates an embodiment of a urinary catheter 10, which may be an intermittent urinary catheter. Urinary catheter 10 includes a urinary catheter tube or shaft 12 having a catheter tube proximal insertion end 14 and a catheter tube distal end 16. A drainage member 20 is associated with catheter tube distal end 16. Drainage member 20 is in fluid communication with an internal drainage lumen (not shown) of urinary catheter tube 12. In some embodiments, drainage member 20 may be a connector for attachment to a urine collection bag or it may be a funnel for directing urine to a receptacle, such as a toilet. Catheter tube proximal insertion end 14 terminates at catheter tip 18, which optionally may be opened or closed. Catheter tube proximal insertion end 14 includes one or more drainage openings or eyelets 22. Drainage openings 22 are in fluid communication with the internal drainage lumen of urinary catheter tube 12. In some embodiments, urinary catheter tube 12 may have a lubricious hydrophilic coating on the outer surface that becomes lubricious when hydrated with a hydration medium. Such coating eases insertion of catheter tube 12 into the urethra. In other embodiments, urinary catheter tube 12 may be lubricated with a gel-lubricant applied to the catheter tube.

In use, urinary catheter tube 12 is inserted and advanced through the urethra of a user so that catheter tube proximal insertion end 14 enters the bladder. When catheter tube proximal insertion end 14 enters the bladder, urine flows from the bladder through the one or more drainage openings 22 and through the internal drainage lumen of the catheter tube 12. The urine flows from the internal drainage lumen and out of drainage member 20.

Referring to FIGS. 1-8, catheter tube 10 may include one or more closure elements 24 that open and close the one or more drainage opening 22. In some alternatives, the one or more closure elements 24 may be covers, shields, barriers, flaps, baffles, cuffs, stomates or other suitable closure elements. The one or more closure elements 24 close drainage openings 22 during insertion and withdrawal of catheter tube 12 through the urethra. In some embodiments, closure elements may include a lubricous hydrophilic coating thereon.

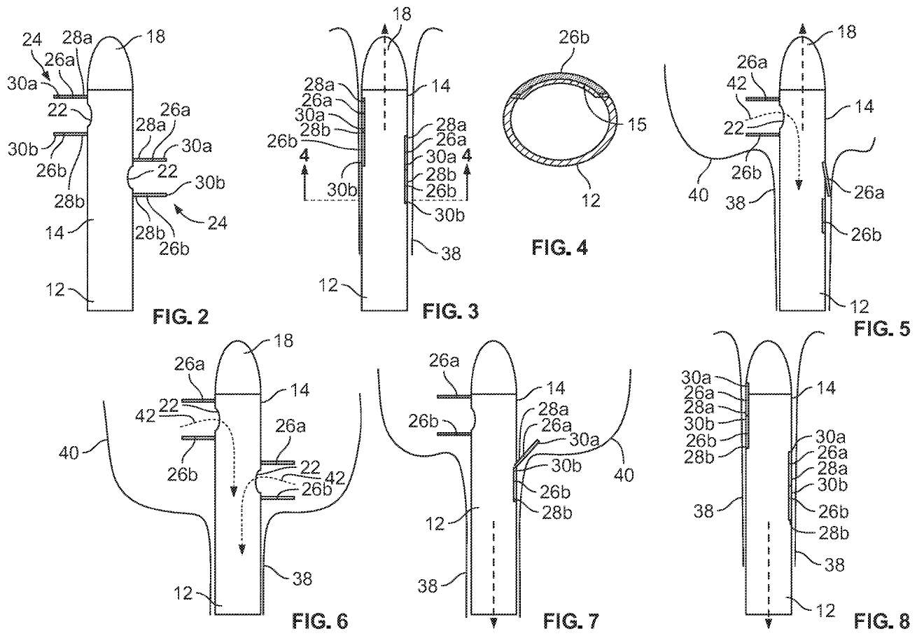

Referring to FIG. 2, in some embodiments, the one or more closure elements 24 are associated with a respective drainage opening 22 and may include a pair of covers 26a, 26b. In the illustrated embodiment, closure elements 24 includes a proximal cover 26a and a distal cover 26b. In the illustrated catheter tube proximal insertion end 14, proximal covers 26a are located proximal of respective drainage opening 22a. Proximal covers 26a includes a first end 28a associated with catheter tube 12. First end 28a may be attached to catheter tube 12 or integral with catheter tube 12. Cover 26a includes a second free end 30a opposite first end 28a. Distal covers 26b are located distal of respective drainage openings 22. Distal cover 26b includes a first end 28b associated with catheter tube 12. First end 28b may be attached to catheter tube 12 or integral with catheter tube 12. Covers 26b include a second free end 30b opposite first end 28b.

Closure elements 24 (e.g., covers 26a, 26b) have an open configuration wherein the one or more drainage openings 22 are exposed or open and a closed configuration wherein the one or more closure elements 24 close or cover the one or more drainage openings 22. For example, referring to FIG. 2, covers 26a, 26b are in the open configuration wherein drainage openings 22 are exposed/open. In this configuration, covers 26a, 26b do not cover drainage openings 22. Covers 26a, 26b extend from the outer surface of catheter tube 12 at an angle so that drainage openings 22 are at least partially exposed/open. In the illustrated embodiment, covers 26a, 26b extend at a 90 degree angle from the outer surface of the catheter tube 12. However, covers 26a, 26b may extend at smaller or large angles in the open configuration as long as the drainage openings 22 are open. In some embodiments, the closure elements 24 (e.g., covers 26a, 26b) are biased toward the open configuration. The closure elements 24 may be made from an elastic material, such as an elastic polymer, that biases the closure elements to the open configuration.

Turning to FIG. 3, when the segment of catheter tube proximal insertion end 14 having closure elements 24 associated therewith is inserted into the urethra, closure elements 24 move into the closed configuration, covering or closing drainage openings 22. Closure elements 24 are movable between the open and closed configurations. In some alternatives, covers 26a, 26b may have a flexible joint between respective first ends 28a, 28b and catheter tube 12. For example, first ends 28a, 28b may be hingedly connected to catheter tube 12. The joints between covers 26a, 26b and catheter tube 12 are bendable to allowing covers 26a, 26b to move between the open configuration and the closed configurations. Covers 26a, 26b have a length between respective first ends 28a, 28b and second free ends 30a, 30b. This length is sufficient to allow one of the covers 26a, 26b to cover the drainage openings 22 in the closed configuration. In some embodiments, the closed configuration may have different states. For example, the closed configuration in a first state may include covers 26a covering or closing respective drainage openings 22 (FIG. 3). In a second state, covers 26b may cover or close respective drainage openings 22 (FIG. 8).

Referring to FIG. 4, in some embodiments covers 26a, 26b may have a curved cross-sectional shape. In some embodiments, the cross-sectional shape may be between semi-circular and a quarter of a circle or less. In some embodiments, the curvature of the cross-sectional shape may be commensurate with the outer curvature of catheter tube 12. Furthermore, optionally, the outer surface of catheter tube 12 may include indentations or depressions 15 which receive covers 26a, 26b in the closed configuration. In the closed configuration, depressions 15 in catheter tube 12 receive respective cover 26a, 26b so that the outer surface of the cover and the outer surface of catheter tube 12 align to form a smooth transition between the outer surfaces. Covers 26a, 26b have a wall thickness between the inner surface and the outer surface of the covers. The wall thickness may be between about 0.3 mm and about 2 mm. In some embodiments, the wall thickness is thinner at free end 30a, 30b than it is at the connected end 28a, 28b.

Turning back to FIGS. 2, 3 and 5-8, covers 26a, 26b are initially in the open configuration (FIG. 2) prior to catheter tube proximal insertion end 14 being inserted into the urethra 38. Referring to FIG. 3, when catheter tube proximal insertion end 14 is inserted into the urethra 38, covers 26a bend or fold distally about the joint between the end 28a and the outer surface of catheter tube 12 so that covers 26a close or cover respective drainage openings 22, thereby moving into the closed configuration. In some embodiments, where covers 26a close drainage openings 22, this is a first closed state of the closed configuration. Similarly, covers 26b bend or fold distally about the joint between end 28b and the outer surface of catheter tube 12 so that covers 26b lay against the outer surface of catheter tube 12. As discussed above, in some optional embodiments, the outer surface of catheter tube 12 includes depressions or indentations 15 that receive the covers 26b. Contact between the inner surface of the urethra 38 and covers 26a, 26b constrains or compresses covers 26a, 26b into the closed configuration. In the closed configurations, in some embodiments, the joints may be rounded or curved so smoothly slide along the inner surface of the urethra 38 during advancement and withdrawal of catheter tube 12 through the urethra.

Turning to FIGS. 5 and 6, when catheter tube proximal insertion end 14 enters bladder 40, covers 26a, 26b move to the open configuration, allowing urine 42 to flow through drainage openings 22 and into the inner drainage lumen of catheter tube 12. Referring to FIGS. 7 and 8, after urine 42 has been drained from bladder 40, catheter tube proximal insertion end 14 is withdrawn from bladder 40 and back into urethra 38. As catheter tube proximal insertion end 14 is withdrawn from bladder 40, the covers 26a, 26b are moved into the closed configuration. Covers 26b bend or fold about the joint between end 28b and the outer surface of the catheter tube 12 so that covers 26b close or cover respective drainage openings 22. In some embodiments, where covers 26b close drainage openings 22, this is a second closed state of the closed configuration. Similarly, covers 26a bend or fold proximally about the joint between end 28a and the outer surface of catheter tube 12 so also to lay against the outer surface of the catheter tube. In comparing FIG. 3 and FIG. 8, in the first closed state of the closed configuration, covers 26a, 26b bend or fold distally (FIG. 3), and in the second closed state of the closed configuration, covers 26a, 26b bend or fold proximally (FIG. 8).

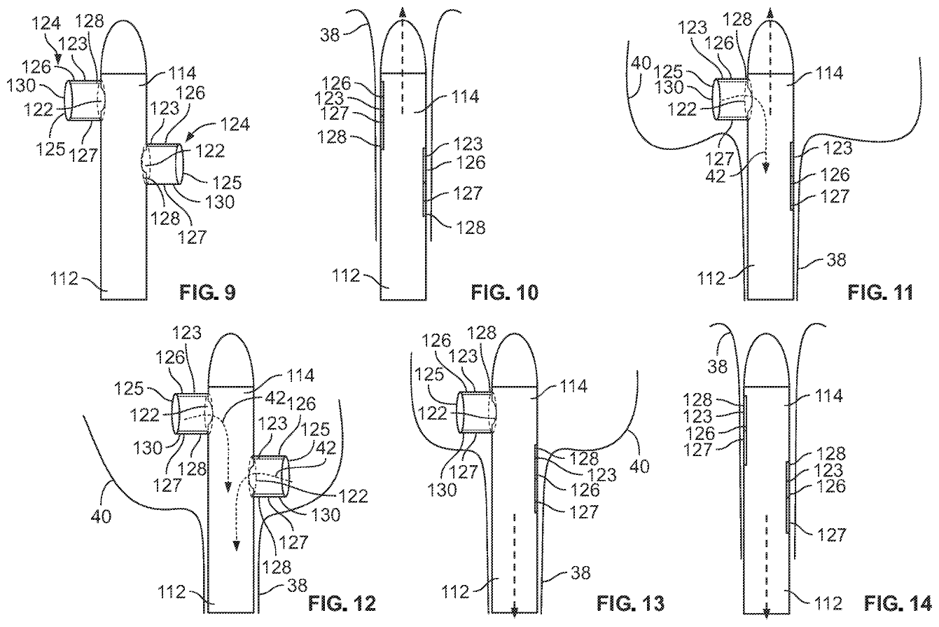

Turning to FIG. 9, in some alternative embodiments, the catheter tube 112 has closure elements 124, which may be cuffs 126. Catheter tube proximal insertion end 114 includes one or more drainage openings 122. Cuffs 126 include a wall 125 that surrounds drainage openings 122. Cuffs 126 may be made from elastic material, such as an elastic polymer. In the illustrated embodiment, cuffs 126 have a generally round cross-section wherein wall 125 is a circumferential wall that surrounds drainage opening 122. However, in some embodiments, the cross-sectional shape of cuffs 126 could be other shapes as well, such as polygonal or irregular shapes.

Cuffs 126 includes an end 128 connected to catheter tube 112 and in communication with openings 122. Cuffs 126 have a free end 130 opposite the attached end 128 and a lumen extending therethrough and in communication with the drainage opening 122. Cuff wall 125 has a proximal portion 123 and a distal portion 127. Cuffs 126 have an open configuration wherein the one or more drainage openings 122 are exposed or open and a closed configuration wherein cuffs 126 close or cover the one or more drainage openings 122. Cuffs 126 may be biased toward the open configuration. FIG. 9 shows cuffs 126 in the open configuration wherein cuff wall 125 extends from the outer surface of catheter tube 112 and does not cover drainage openings 122. In the closed configuration, cuffs 126 bend or fold so that cuff wall 125 closes or covers drainage opening 122. Similar to the catheter tube 12, catheter tube 112 may have indentations or depressions that receive portions of cuffs 126 so as to have a smooth transition between the folded cuff wall 125 and the outer surface of the catheter.

Referring to FIGS. 9-14, cuffs 126 are initially in the open configuration (FIG. 9) prior to catheter tube proximal insertion end 114 being inserted into the urethra. Referring to FIG. 10, when catheter tube proximal insertion end 114 is inserted into the urethra, cuffs 126 contact the urethra and bend or fold distally about the joint between end 128 and the outer surface of catheter tube 112 so that cuff walls 125 close or cover respective drainage openings 122. In the illustrated embodiment, proximal portion 123 of cuff wall 125 covers or closes drainage opening 122, and distal portion 127 lays on the outer surface of catheter tube 112. In some embodiments, this is the first closed state of the closed configuration. The inner wall of urethra 38 compresses or constrains cuffs 126 so that they remain in the closed configuration.

Turning to FIGS. 11 and 12, when catheter tube proximal insertion end 114 enters the bladder 40, cuffs 126 move from the closed configuration to the open configuration, allowing urine 42 to flow through drainage openings 122 and into the inner drainage lumen of catheter tube 112. Referring to FIGS. 13 and 14, after urine 42 has been drained from the bladder 40, catheter tube proximal insertion end 114 is withdrawn from bladder 40 and back into the urethra 38. As catheter tube proximal insertion end 114 is withdrawn from bladder 40, cuffs 126 are moved into the closed configuration. Cuffs 126 bend or fold about the joint between end 128 and the outer surface of catheter tube 112 so that distal portion 127 of cuff wall 125 closes or covers respective drainage openings 122 and proximal portion 123 of cuff wall 125 lays on the outer surface of catheter tube 112. In some embodiments, this is the second closed state of the closed configuration.

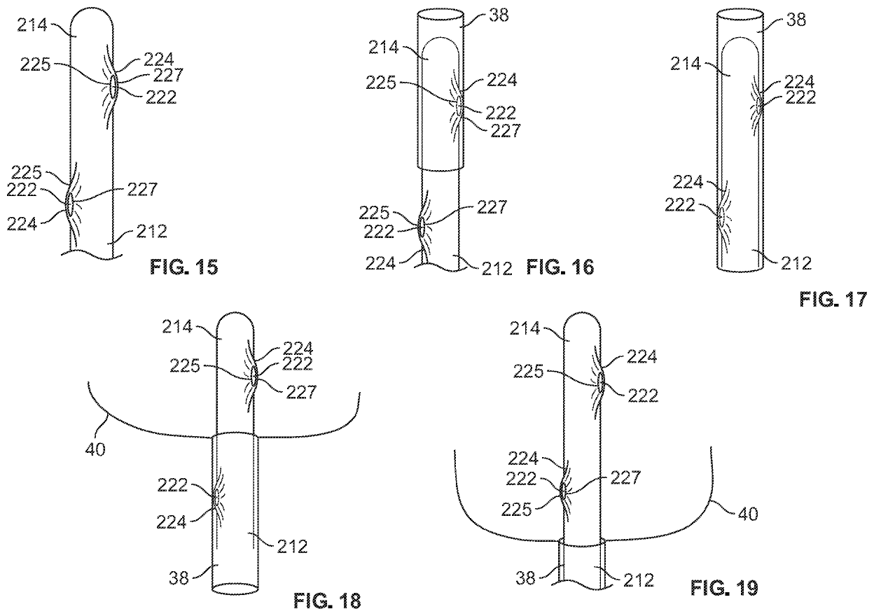

Turning to FIGS. 15-19, proximal insertion end 214 of catheter tube 212 includes one or more drainage openings 222. In some embodiments, openings 222 include closure elements, such as stomata closures 224 (which includes stomata-like closures) that open and close drainage openings 222. The opening and closing of stomata closures 224, including stomata-like closures, depends on whether catheter tube proximal insertion end 214 is unconstrained or constrained. Referring to FIG. 15, stomata closures 224 have an open configuration when catheter proximal insertion end 214 is unconstrained and stomata closures 224 are open. Referring to FIG. 16, stomata closures 224 have a closed configuration when catheter proximal insertion end 214 is constrained and stomata closures 224 are closed.

In the illustrated embodiment, stomata closures 224 have a first flexible edge 225 and an opposed second opposed flexible edge 227 defining the drainage opening 222. In the open configuration, when catheter tube proximal insertion end 214 is unconstrained, first flexible edge 225 and second flexible edge 227 are separated or apart, thereby opening drainage opening 222 (FIGS. 15 and 19). When catheter tube proximal insertion end 214 is constrained, for example by radial inward pressure from urethra 38, first flexible edge 225 and second flexible edge 227 move together and abut to close drainage opening 222 (FIG. 17).

In some embodiments, stomata closures 224 of drainage openings 222 may be formed by thermoforming-vacuum forming, blow molding or injection molding (this may include forming the catheter and the eyelet in a single step). For example, stomata closures 224 may be formed by mechanical punching (without a mandrel), thermo-forming (hot eye forming), skiving, drilling, laser cutting etc. Although the illustrated embodiment shows an “l” shaped stomata closure (e.g., narrow oval), the stomata closure may also be an “X” or “Y” shape depending on desired use. The stomata closure shapes could be formed with smooth/annealed edges by using a hot eye forming approach. In some embodiments, the stomata edges that define the drainage opening may be finished with a smooth (annealed) or polished edge and may further have a lubricious surface. Furthermore, stomata closures 224 may be in a staggered sequence on catheter tube 212 or directly opposite one another.

In some embodiments, catheter tube proximal insertion end 214 may have a larger diameter than the rest of the main body of catheter tube 212. In some embodiments, at least a portion of the catheter proximal insertion end 214 has an outer diameter that is sufficiently large so that the portion of the catheter proximal insertion end 214 is compressed by the urethra. In some embodiments, the outer diameter of at least a portion of catheter proximal insertion end 214 is larger than the inner diameter of the urethra. Additionally, catheter proximal end 214 may have softer and/or more flexible material or structures in the region of stomata closures 224 than in other portions of the catheter. The softer material or structures may assist in moving stomata closures 224 between the open and closed configurations as catheter tube proximal insertion end 214 is compressed (constrained) and uncompressed (unconstrained).

Referring to FIGS. 15-19, stomata closures 224 of drainage openings 222 are initially in the open configuration (FIG. 15) and are open prior to the catheter tube proximal insertion end 214 being inserted into the urethra. As mentioned above, stomata closures 224 may be biased to the open configuration. Referring to FIGS. 16 and 17, when catheter tube proximal insertion end 214 is inserted into urethra 38, the urethra compresses or constrains catheter tube proximal insertion 214, moving stomata closures 224 from the open configuration to the closed configuration in which the closures are closed. In the illustrated embodiment, stomata edges 225 and 227 move together to abut, thereby closing stomata closures 224, which remain closed as the catheter tube 212 is advanced through the urethra.

Turning to FIGS. 18 and 19, when catheter tube proximal insertion end 214 enters bladder 40, it is uncompressed or unconstrained and stomata closures 224 move into the open configuration. In the open configuration, stomata closures edges 225, 227 move apart to open drainage openings 222 and allow urine to enter drainage openings 222 to drain urine from bladder 40. After urine has been drained from bladder 40, catheter tube proximal insertion end 214 is withdrawn from bladder 40 and back into urethra 38. As catheter tube proximal insertion end 214 is withdrawn from bladder 40, stomata closures 224 are moved into the closed configuration wherein stomata closures closed drainage openings 222.

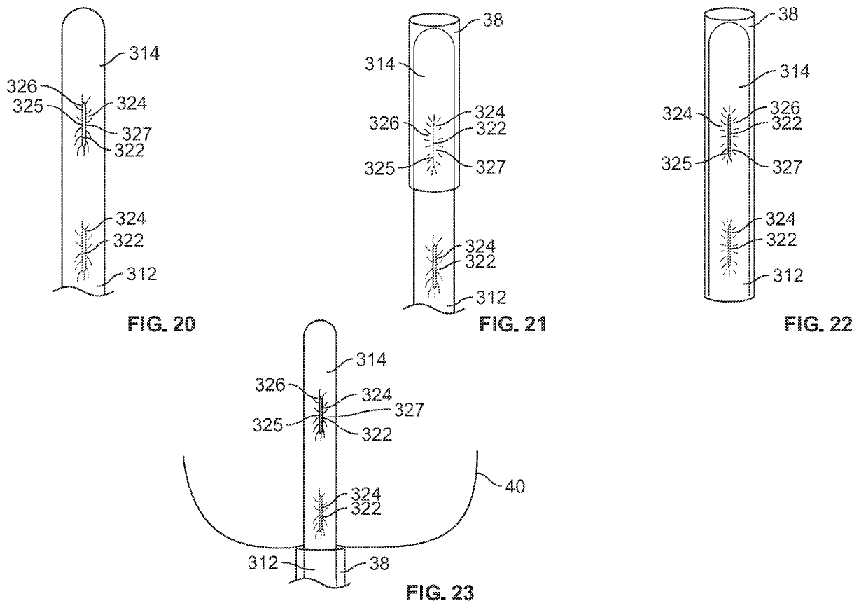

Turning to FIGS. 20-23, there is shown a proximal insertion end 314 of catheter tube 312 that includes alternative drainage openings 322 having stomata closures 324 that open and close drainage openings 322. The opening and closing of stomata closures 324 depends on whether catheter tube proximal insertion end 314 is unconstrained or constrained. Referring to FIG. 20, stomata closures 324 have an open configuration when catheter proximal insertion end 314 is unconstrained and stomata closures 324 are open to open drainage openings 322. Referring to FIG. 22, stomata closures 324 have a closed configuration when catheter tube proximal insertion end 314 is constrained and stomata closures 324 are closed, thereby closing drainage openings 322.

In the illustrated embodiment, stomata closures 324 are elongated in the direction of the elongated axis of catheter tube 312. Stomata closures 324 have a first flexible edge 325 and an opposed second opposed flexible edge 327 defining drainage opening 322. The portion or region 326 of material about stomata closures 324 may be elastic and flexible. In the open configuration, when catheter tube proximal insertion end 314 is unconstrained, first flexible edge 325 and second flexible edge 327 are separated or apart, thereby opening drainage opening 322. In some embodiments, stomata closures 324 are biased to the open position. When catheter tube proximal insertion end 314 is constrained, for example by radial inward pressure from urethra 38, first flexible edge 325 and second flexible edge 327 move together and abut to close drainage opening 322.

Referring to FIGS. 20-23, stomata closures 324 of drainage openings 322 are initially in the open configuration (FIG. 20) and are open prior to the catheter tube proximal insertion end 314 being inserted into the urethra. As mentioned above, stomata closures 324 may be biased to the open configuration. Referring to FIGS. 21 and 21, when catheter tube proximal insertion end 314 is inserted into urethra 38, the urethra compresses or constrains catheter tube proximal insertion 314, moving stomata closures 324 from the open configuration to the closed configuration in which the closures are closed. In the illustrated embodiment, stomata edges 325 and 327 move together to abut, thereby closing stomata closures 324, which remain closed as the catheter tube 312 is advanced through the urethra.

Turning to FIG. 23, when catheter tube proximal insertion end 314 enters bladder 40, it is uncompressed or unconstrained and stomata closures 324 move into the open configuration. In the open configuration, stomata closures edges 325, 327 move apart to open drainage openings 322 and allow urine to enter drainage openings 322 to drain urine from bladder 40. After urine has been drained from bladder 40, catheter tube proximal insertion end 314 is withdrawn from bladder 40 and back into urethra 38. As catheter tube proximal insertion end 314 is withdrawn from bladder 40, stomata closures 324 are moved into the closed configuration wherein stomata closures closed drainage openings 322.

It should be understood that various changes and modifications to the presently preferred embodiments described herein will be apparent to those skilled in the art. Such changes and modifications can be made without departing from the spirit and scope of the invention disclosed herein.

What is claimed is:

1. A urinary catheter product, comprising:
a catheter tube configured to be inserted into a urethra, the catheter tube having a catheter tube proximal insertion end, a catheter tube distal end and a catheter tube internal drainage lumen;
one or more drainage openings associated with the catheter tube proximal insertion end, the one or more drainage openings in fluid communication with the catheter tube internal drainage lumen and being configured to drain urine from a bladder; and
one or more closure elements associated with the one or more drainage openings, the one or more closure elements having an open configuration wherein the one or more drainage openings are exposed and a closed configuration wherein the one or more closure elements close the one or more drainage openings.

2. The urinary catheter product of claim 1, wherein the one or more closure elements comprise one or more stomata closures.

3. The urinary catheter product of claim 1, wherein the one or more closure elements comprise one or more covers.

4. The urinary catheter product of claim 1, wherein the one or more closure elements comprise cuffs.

5. The urinary catheter product of claim 1, wherein the one or more closure elements cover the one or more drainage openings to close the one or more drainage openings.

6. The urinary catheter product of claim 1, wherein the one or more closure elements are moveable between the open configuration and the closed configuration.

7. The urinary catheter product of claim 1, wherein the one or more closure elements are configured to be in the open configuration when the catheter tube proximal insertion end is unconstrained by the urethra.

8. The urinary catheter product of claim 3, wherein the one or more covers are in the open configuration when the one or more drainage openings are located within the bladder.

9. The urinary catheter product of claim 1, wherein the one or more closure elements are in the closed configuration when the proximal end of the catheter tube is constrained by the urethra.

10. The urinary catheter product of claim 1, wherein the one or more closure elements are in the closed configuration when the one or more drainage openings are located within the urethra.

11. The urinary catheter product of claim 1, wherein the one or more closure elements are made from an elastic material, preferably an elastic polymer.

12. The urinary catheter product of claim 1, wherein the one or more closure elements are initially in the open configuration, move into the closed configuration as the catheter tube is inserted through the urethra, move into back to the open configuration when the one or more drainage openings are in the bladder, and move back into the closed configuration when the catheter tube is withdrawn from the urethra.

13. The urinary catheter of claim 3, wherein the one or more covers comprise a pair of opposed covers, the covers having a first end operatively attached to the catheter tube and a second free end.

14. The urinary catheter of claim 13, wherein the one or more covers have a wall thickness between an outer surface and an inner surface, wherein the thickness is between about 0.3 mm and about 2 mm.

15. The urinary catheter of claim 14, wherein the wall thickness is smaller at the second free end than at the first attached end.

16. The urinary catheter of claim 13, wherein a first cover of the pair of opposed covers is located proximal relative a respective drainage opening and a second cover of the pair of opposed covers is located distally relative to the respective drainage opening.

17. The urinary catheter of claim 3, in which an outer surface of the catheter tube includes one or more depressions which receives the one or more closure elements when the covers are in the closed configuration.

18. The urinary catheter of claim 2, wherein the stomata closure comprises an I, X, or Y shape.

19. The urinary catheter of claim 2, wherein in the one or stomata closures have a first edge and an opposed second edge, wherein the first edge and second edge define one of the one or more openings.

20. The urinary catheter of claim 1, wherein the one or more closure elements are biased to the open configuration.
